(19) United States
(12) Patent Application Publication
(10) Pub. No.: US 2004/0265346 A1
(43) Pub. Date: Dec. 30, 2004

(54) Water-in-oil emulsions for use in cosmetics

(76) Inventors: Aurore Verloo, Bievres (FR); Jacqueline Fontaine, La Celle Saint Cloud (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/833,165

(22) Filed: Apr. 28, 2004

Related U.S. Application Data
(60) Provisional application No. 60/467,580, filed on May 5, 2003.

(30) Foreign Application Priority Data
Apr. 28, 2003 (FR) 03 05175

(51) Int. Cl. A61K 7/00; G03C 5/18; G03C 5/26

(52) U.S. Cl. 424/401

(57) ABSTRACT

A water-in-oil solid emulsion comprising an aqueous phase emulsified with at least one emulsifying surfactant, wherein said at least one emulsifying surfactant is in a fatty phase comprising at least one oil, at least one wax, and at least one block copolymer comprising at least one first monomeric unit chosen from ethylene oxide and propylene oxide units and at least one second monomeric unit chosen from alkylene oxide units comprising from 6 to 40 carbon atoms, wherein the at least one block copolymer has a weight-average molecular weight ranging from 5000 g/mol to 8000 g/mol and the emulsion has a hardness such that the penetration force is greater than or equal to 40 g. Also disclosed herein is the use of this emulsion for making up and/or caring for the skin.

This application claims benefit of U.S. Provisional Application No. 60/467,580, filed May 5, 2003.

Disclosed herein is a water-in-oil solid emulsion comprising an aqueous phase emulsified with at least one emulsifying surfactant in a fatty phase comprising at least one oil, at least one wax; and at least one particular block copolymer, wherein the emulsion has a hardness such that the penetration force is greater than or equal to 40 g.

Further disclosed herein is a process for making up and/or caring for human skin, comprising applying the water-in-oil solid emulsion to the skin.

Skin makeup products such as foundations are known in very diverse galenical forms: free powders, compact powders, cast solid products, sticks, and fluid creams. The compact powders generally have a very matte appearance, whereas the fluid creams may have a rather shiny appearance.

The cast solid products may be anhydrous or may be in the form of emulsions. These emulsions generally contain fatty substances such as oils and solid waxes, water and a particulate phase generally comprising fillers and pigments, as described, for example, in patent application Publication No. WO 99/47111.

The solid emulsion does not flow under its own weight at room temperature, and is suitable for packaging in a case. To apply the product, the user can take up the product directly by disintegrating it using the fingers or an applicator such as a sponge.

However, the presence of wax in the solid emulsion may have a tendency to opacify the product. As a result, the product may have a matte surface appearance. Furthermore, when the user takes up the product with the fingers or with a sponge by disintegrating the surface of the solid composition, he or she may notice a certain dragging effect during the disintegration. This dragging effect may occur because the product is not sufficiently slippery to facilitate rapid uptake of the product.

Thus, it would be useful to provide a solid makeup or skincare composition that has good slipperiness when the product is taken up with the fingers or with a sponge, facilitating a rapid uptake of the product during its disintegration. It would also be useful to provide a solid composition that has a shiny appearance during its disintegration.

The inventors have discovered that by using the disclosed water-in-oil solid emulsion, it is possible to obtain a solid product that may have good slipperiness during its disintegration and that may allow the user to satisfactorily take up the product with the fingers or with a sponge. Furthermore, the product before use may have a matte surface appearance, which becomes shiny during its disintegration: thus, the solid product may have a mirror appearance during disintegration. This visual mirror effect makes the product very appealing to users.

The emulsion disclosed herein is a water-in-oil solid emulsion comprising an aqueous phase emulsified with at least one emulsifying surfactant, wherein said emulsifying surfactant is in a fatty phase comprising at least one oil, at least one wax; and at least one block copolymer comprising at least one first monomeric unit chosen from ethylene oxide and propylene oxide units and at least one second monomeric unit chosen from alkylene oxide units comprising from 6 to 40 carbon atoms, wherein the at least one block copolymer has a weight-average molecular weight ranging from 5000 g/mol to 8000 g/mol and wherein the emulsion has a hardness such that the penetration force is greater than or equal to 40 g.

Further disclosed herein is a non-therapeutic cosmetic makeup and/or care process for the skin, comprising applying to the skin the at least one water-in-oil solid emulsion disclosed herein.

As used herein, the term “solid emulsion” means an emulsion that does not flow under its own weight at room temperature (25° C.) after 1 hour.

The water-in-oil solid emulsion disclosed herein comprises at least one block copolymer comprising at least one first monomeric unit chosen from ethylene oxide and propylene oxide units and at least one second monomeric unit chosen from alkylene oxide units comprising from 6 to 40 carbon atoms, wherein the at least one block copolymer has a weight-average molecular weight ranging from 5000 g/mol to 8000 g/mol.

For example, the alkylene oxide units of the at least one block copolymer may comprise from 6 to 30 carbon atoms, for example, from 8 to 20 carbon atoms, further, for example, from 10 to 18 carbon atoms and, even further, for example, from 10 to 14 carbon atoms.

The weight-average molecular weight of the at least one block copolymer ranges from 5000 g/mol to 8000 g/mol, for example, from 5500 g/mol to 7000 g/mol, further, for example, from 5500 g/mol to 6500 g/mol and, even further, for example, from 5800 g/mol to 6200 g/mol.

The at least one block copolymer may, for example, comprise from 35 to 55 first monomeric units chosen from ethylene oxide and propylene oxide units and from 15 to 30 second monomeric units chosen from alkylene oxide units comprising from 6 to 40 carbon atoms.

For example, the at least one block copolymer is such that the ratio of the number of ethylene oxide and/or propylene oxide units to the number of alkylene oxide units comprising from 6 to 40 carbon atoms ranges from 1.5:1 to 2.5:1, for example, from 1.8:1 to 2.3:1 and, further, for example, from 1.9:1 to 2.1:1.

Such copolymers are described, for example, in Patent No. FR-A-2 425 848 and sold under the name ELFACOS ST 9 by the company Akzo Nobel.

The at least one block copolymer may be present in the water-in-oil emulsion disclosed herein in an amount ranging from 1% to 25% by weight, further, for example, ranging from 1% to 18% by weight and, even further, for example, ranging from 1% to 10% by weight, relative to the total weight of the emulsion.

The fatty phase of the emulsion disclosed herein comprises at least one oil, which may be chosen from oils of mineral, animal, plant and synthetic origin, carbon-based oils, hydrocarbon-based oils, fluoro oils and silicone oils.

As used herein, the term “hydrocarbon-based oil” means an oil comprising carbon and hydrogen atoms, and optionally oxygen and nitrogen atoms, and comprising no silicon or fluorine atoms. It may comprise at least one group chosen from alcohol, ester, ether, carboxylic acid, amine and amide groups.

As used herein, the term “silicone oil” means an oil comprising at least one silicon atom and, for example, comprising at least one Si—O group.

As used herein, the term “fluoro oil” means an oil comprising at least one fluorine atom.

In one embodiment, the emulsion disclosed herein may comprise at least one oil chosen from volatile oils. The term “volatile oil” means an oil (or a non-aqueous medium) that is capable of evaporating on contact with the skin in less than 1 hour, at room temperature and atmospheric pressure. The volatile oils are chosen from volatile cosmetic oils, which are liquid at room temperature, for example, having a non-zero vapor pressure, at room temperature and atmospheric pressure, for example, having a vapor pressure ranging from 0.13 Pa to 40 000 Pa (10⁻³ to 300 mmHg) and, further for example, ranging from 1.3 Pa to 13 000 Pa (0.01 to 100 mmHg) and, even further, for example, ranging from 1.3 Pa to 1300 Pa (0.01 to 10 mmHg).

In addition, the volatile oils generally have a boiling point, measured at atmospheric pressure, ranging from 150° C. to 260° C. and, for example, ranging from 170° C. to 250° C.

The volatile oils may, for example, be chosen from at least one of:

linear and cyclic silicone oils with a viscosity at room temperature of less than 8 mm²/s and, for example, comprising from 2 to 7 silicon atoms, these silicones optionally comprising at least one group chosen from alkyl and alkoxy groups comprising from 1 to 10 carbon atoms. Examples of volatile silicone oils that may be used in the emulsion disclosed herein include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane;

hydrocarbon-based volatile oils comprising from 8 to 16 carbon atoms and mixtures thereof, and, for example, branched C₈-C₁₆ alkanes, such as C₈-C₁₆ isoalkanes (also known as isoparaffins), isododecane, isodecane, isohexadecane and, for example, the oils sold under the trade names ISOPAR and PERMETHYL, branched C₈-C₁₆ esters, for example, isohexyl neopentanoate, and mixtures thereof; isododecane may, for example, be used; and

volatile fluoro oils such as nonafluoroethoxybutane, nonafluoromethoxybutane, decafluoropentane, tetradecafluorohexane or dodecafluoropentane, and mixtures thereof.

In one embodiment, the emulsion disclosed herein comprises at least one oil chosen from volatile silicone oils.

In one embodiment, the volatile oils may be present in an amount ranging from 0.1% to 35% by weight, for example, ranging from 1% to 30% by weight, and, further, for example, ranging from 5% to 25% by weight, relative to the total weight of the emulsion.

The emulsion may also comprise at least one oil chosen from non-volatile oils.

The non-volatile oils may, for example, be chosen from at least one of the following oils:

hydrocarbon-based oils of mineral and synthetic origin, such as linear and branched hydrocarbons, for example, liquid paraffin and derivatives thereof, liquid petroleum jelly, polydecenes, hydrogenated polyisobutene such as PARLEAM sold by the company Nippon Oil Fats, and squalane of synthetic and plant origin;

oils of animal origin, for example, mink oil, turtle oil and perhydrosqualene;

hydrocarbon-based oils of plant origin with a high triglyceride content comprising fatty acid esters of glycerol, the fatty acids of which may comprise at least one chain chosen from chains with varying chain lengths, wherein the at least one chain is chosen from linear and branched, and saturated and unsaturated chains, such as fatty acid triglycerides comprising, for example, from 4 to 22 carbon atoms, for example, heptanoic and octanoic acid triglyceride, and capric/caprylic acid triglyceride, and hydroxylated triglycerides, such as sweet almond oil, beauty-leaf oil, palm oil, grapeseed oil, sesame oil, arara oil, rapeseed oil, sunflower oil, cotton oil, apricot oil, castor oil, alfalfa oil, marrow oil, blackcurrant oil, macadamia oil, musk rose oil, hazelnut oil, avocado oil, jojoba oil, olive oil, cereal (maize, wheat, barley and rye) germ oil, and shea butter;

fatty acid esters, comprising, for example, from 4 to 22 carbon atoms, and, for example, fatty acid esters of octanoic acid, of heptanoic acid, of lanolic acid, of oleic acid, of lauric acid or of stearic acid, such as propylene glycol dioctanoate, propylene glycol monoisostearate, polyglyceryl-2 diisostearate and neopentyl glycol diheptanoate;

synthetic esters of formula R₁COOR₂ wherein R₁ is chosen from linear and branched higher fatty acid residues comprising from 7 to 40 carbon atoms and R₂ is chosen from branched hydrocarbon-based chains comprising from 3 to 40 carbon atoms, such as purcellin oil (cetostearyl octanoate), isononyl isononanoate, C₁₂ to C₁₅ alkyl benzoate, 2-ethylhexyl palmitate, 2-octyldodecyl stearate, 2-octyidodecyl erucate, isostearyl isostearate, 2-octyidodecyl benzoate, alkyl and polyalkyl octanoates, decanoates and ricinoleates, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate and isodecyl neopentanoate;

hydroxylated esters, for example, isostearyl lactate, octyl hydroxystearate, octyidodecyl hydroxystearate, diisostearyl malate, triisocetyl citrate, glyceryl triisostearate and diglyceryl triisostearate; diethylene glycol diisononanoate; pentaerythritolesters; esters of aromatic acids and of alcohols comprising from 4 to 22 carbon atoms, for example, tridecyl trimellitate;

C₈-C₂₆ higher fatty acids such as oleic acid, linoleic acid, linolenic acid and isostearic acid;

C₈-C₂₆ higher fatty alcohols such as oleyl alcohol, linoleyl alcohol, linolenyl alcohol, isostearyl alcohol and octyldodecanol;

synthetic esters comprising at least 7 carbon atoms;

silicone oils such as polydimethylsiloxanes (PDMS) that are liquid at room temperature, linear, and optionally phenylated, such as phenyltrimethicones, phenyltrimethylsiloxydiphenylsiloxanes, diphenyl dimethicones, diphenylmethyld iphenyltrisiloxanes, liquid 2-phenylethyl trimethylsiloxysilicates, optionally substituted with at least one group chosen from aliphatic and aromatic groups, such as alkyl, alkoxy and phenyl groups, wherein the at least one group is pendent and/or at the end of a silicone chain, the at least one group comprising from 2 to 24 carbon atoms and being optionally fluorinated, or substituted with at least one functional group, for example, chosen from hydroxyl, thiol and amine groups;

polysiloxanes modified with fatty acids, fatty alcohols or polyoxyalkylenes, for example, dimethicone copolyols and alkylmethicone copolyols; and

liquid fluorosilicones.

For example, the non-volatile oils may be present in an amount ranging from 0.1% to 35% by weight, for example, ranging from 1% to 30% by weight, and even further, for example, ranging from 5% to 25% by weight, relative to the total weight of the emulsion.

The emulsion disclosed herein has a hardness such that the penetration force is greater than or equal to 40 grams (g), for example, ranging from 40 g to 150 g.

The penetration force of the emulsion is measured according to the following protocol:

At the end of preparation of the emulsion, it is cast into a dish and maintained at 20° C. for 24 hours. The penetration force is then measured on this solid emulsion using a Stevens texture analyser machine, with a TA 24 measuring spindle, 4 mm in diameter, at a penetration speed of 0.5 mm/s and to a preselected penetration depth of 2 mm. The penetration force, expressed in grams, is read on the machine.

For example, the solid emulsion has a hardness such that the penetration force ranges from 40 g to 150 g, for example, from 50 g to 120 g, further, for example, from 50 g to 90 g and, even further, for example, from 60 g to 80 g.

In one embodiment, the at least one wax present in the emulsion disclosed herein may be chosen from waxes with a hardness ranging from 5 MPa to 9 MPa, for example, ranging from 6 MPa to 9 MPa and, further, for example, ranging from 7 MPa to 9 MPa.

As used herein, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The hardness of the wax is determined by measuring the compressions force, measured at 20° C. using a texturometer sold under the name TA-XT2i by the company Rheo, equipped with a stainless-steel cylinder 2 mm in diameter travelling at a measuring speed of 0.1 mm/s, and penetrating into the wax to a penetration depth of 0.3 mm. To perform the hardness measurement, the wax is melted at a temperature equal to the melting point of the wax +20° C. The molten wax is poured into a container 30 mm in diameter and 20 mm deep. The wax is recrystallized at room temperature (25° C.) for 24 hours and is then stored for at least 1 hour at 20° C. before performing the hardness measurement. The hardness value is the measured compression force divided by the surface area of the texturometer cylinder in contact with the wax.

As used herein, the at least one wax may be chosen from hydrocarbon-based waxes, silicone waxes and fluoro waxes, optionally comprising at least one functional group chosen from ester and hydroxyl functional groups.

The at least one wax having the hardness as defined above may be chosen from carnauba wax, microcrystalline waxes, ozokerites, hydrogenated jojoba oil, polyethylene waxes such as the wax sold under the name PERFORMALENE 400 Polyethylene by the company New Phase Technologies, silicone waxes, for example, poly(C₂₄-C₂₈)alkylmethyldimethylsiloxane, such as the product sold under the name ABIL WAX 9810 by the company Goldschmidt, palm butter, the C₂₀-C₄₀ alkyl stearate sold under the name KESTER WAX K82H by the company Kester Keunen, stearyl benzoate, and shellac wax. In one embodiment, the at least one wax may be chosen from carnauba wax, ozokerites, hydrogenated jojoba oil and polyethylene waxes.

The at least one wax may be present in the emulsion disclosed herein in an amount ranging from 1 % to 10% by weight, for example, ranging from 2% to 7% by weight and, even further, for example, ranging from 2.5% to 5% by weight, relative to the total weight of the emulsion.

The aqueous phase of the solid emulsion disclosed herein comprises water. The water may be chosen from at least one of floral water such as cornflower water, mineral water such as eau de Vittel, eau de Lucas and eau de La Roche Posay, and spring water.

The aqueous phase may also comprise at least one organic solvent that is miscible with water (at room temperature—25° C.), for example, chosen from C₂-C₄ primary alcohols such as ethanol and isopropanol, glycols such as propylene glycol, butylene glycol, dipropylene glycol, diethylene glycol, glycol ethers, such as C₁-C₄ alkyl ethers of mono-, di- and tripropylene glycol, and mono-, di- and triethylene glycol.

The aqueous phase may also comprise at least one stabilizer, for example, chosen from sodium chloride, magnesium dichloride and magnesium sulphate.

The aqueous phase may also comprise at least one water-soluble or water-dispersible compound that is compatible with an aqueous phase, for example, chosen from gelling agents, film-forming polymers, thickeners and surfactants.

For example, the aqueous phase may be present in the emulsion disclosed herein in an amount ranging from 30% to 50% by weight and, for example, ranging from 35% to 45% by weight, relative to the total weight of the emulsion.

The emulsion disclosed herein also comprises at least one emulsifying surfactant allowing the production of a water-in-oil emulsion, for example, chosen from surfactants with an HLB (hydrophilic/lipophilic balance) value of less than 7. For example, the at least one emulsifying surfactant may be chosen from fatty acid esters of polyols, such as glyceryl and sorbitol mono-, di-, tri- and sesquioleates and stearates, and glyceryl and polyethylene glycol laurates; alkyl and alkoxy dimethicone copolyols comprising at least one chain chosen from alkyl and alkoxy chains, wherein the at least one chain is pendent or at the end of a silicone skeleton and for example, comprises from 6 to 22 carbon atoms; and polyoxyalkylenated fatty acid esters of glycol.

For example, the at least one emulsifying surfactant may be a C₈-C₂₂ alkyl dimethicone copolyol, i.e. an oxypropylenated and/or oxyethylenated polymethyl(C₈-C₂₂)-alkyldimethylmethylsiloxane.

The C₈-C₂₂ alkyl dimethicone copolyol may, for example, be a compound of formula (I) below:

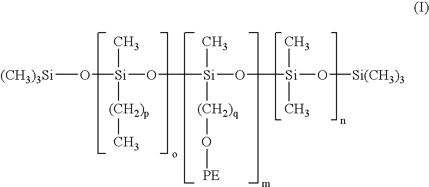

wherein:

PE is chosen from groups (—C₂H₄O)ₓ—(C₃H₆O)_{y}—R, wherein R is chosen from a hydrogen atom and alkyl radicals comprising from 1 to 4 carbon atoms, x is a number ranging from 0 to 100 and y is a number ranging from 0 to 80, provided that x and y are not simultaneously equal to 0;

m is a number ranging from 1 to 40;

n is a number ranging from 10 to 200;

o is a number ranging from 1 to 100;

p is a number ranging from 7 to 21; and

q is a number ranging from 0 to 4.

and, for example:

R is a hydrogen atom;

m is a number ranging from 1 to 10;

n is a number ranging from 10 to 100;

o is a number ranging from 1 to 30;

p is equal to 15; and

q is equal to 3.

Examples of C₈-C₂₂ alkyl dimethicone copolyols include cetyl dimethicone copolyol such as the product sold under the name Abil EM-90 by the company Goldschmidt.

Other examples of the at least one emulsifying surfactant that may be used to obtain a W/O emulsion include polymers such as polyoxyalkylenated fatty acid esters of glycol with water-in-oil emulsifying properties.

The fatty acid ester of the polymer may, for example, be polyhydroxylated. For example, the polymer may be a block polymer, for example, having an ABA structure, comprising poly(hydroxylated ester) blocks and polyethylene glycol blocks.

The fatty acid ester of the emulsifying polymer defined above generally has at least one chain comprising from 12 to 20 carbon atoms, for example, from 14 to 18 carbon atoms. The esters may be chosen, for example, from oleates, palmitates and stearates.

The polyethylene glycol blocks of the emulsifying polymer as defined above may, for example, comprise from 4 to 50 mol of ethylene oxide and, further, for example, from 20 to 40 mol of ethylene oxide.

One polymer surfactant that may be suitable for producing the emulsions disclosed herein is 30 EO polyethylene glycol dipolyhydroxystearate, sold under the trade name ARLACEL P 135 by the company ICI.

The at least one emulsifying surfactant may be present in the emulsion disclosed herein in an amount ranging from 1% to 10% by weight and, for example, ranging from 2% to 5% by weight, relative to the total weight of the composition.

The emulsion disclosed herein may further comprise at least one filler.

As used herein, the term “fillers” means colorless or white, mineral or synthetic particles of any shape, which are insoluble in the medium of the emulsion irrespective of the temperature at which the emulsion is manufactured.

The at least one filler may be chosen from mineral and organic fillers of any shape, in platelet form, spherical and oblong, irrespective of the crystallographic shape (for example, lamellar, cubic, hexagonal, orthorhombic, etc.). For example, the at least one filler may be chosen from talc, mica, silica, kaolin, powders of polyamide (NYLON), of poly-β-alanine and of polyethylene, powders of tetrafluoroethylene polymers (TEFLON), lauroyllysine, starch, boron nitride, hollow polymer microspheres such as those of polyvinylidene chloride/acrylonitrile, for example, EXPANCEL (Nobel Industrie), acrylic acid copolymers, silicone resin microbeads (for example TOSPEARLS from Toshiba), polyorganosiloxane elastomer particles, precipitated calcium carbonate, magnesium carbonate and magnesium hydrocarbonate, hydroxyapatite, hollow silica microspheres, glass and ceramic microcapsules, metal soaps derived from organic carboxylic acids comprising from 8 to 22 carbon atoms and, for example, from 12 to 18 carbon atoms, for example, zinc stearate, magnesium stearate, lithium stearate, zinc laurate and magnesium myristate.

The at least one filler may be present in the emulsion in an amount ranging from 0.1% to 25% by weight and, for example, from 1% to 15% by weight, relative to the total weight of the emulsion.

The emulsion disclosed herein may further comprise at least one dyestuff chosen from water-soluble and liposoluble dyes, pigments and nacres.

As used herein, the term “pigments” means white or colored, mineral or organic particles, which are insoluble in the liquid organic phase, and which are intended to color and/or opacify the composition.

As used herein, the term “nacres” means iridescent particles, produced, for example, by certain molluscs in their shell or, alternatively, synthesized, which are insoluble in the medium of the composition.

As used herein, the term “dyes” means compounds, for example, organic compounds, which are soluble in fatty substances, such as oils, or in an aqueous-alcoholic phase.

The pigments may be chosen from mineral and organic pigments. For example, the pigments may be chosen from at least one of metal oxides, such as iron oxides (for example, yellow, red, brown and black iron oxide), titanium dioxides, cerium oxide, zirconium oxide and chromium oxide; manganese violet, ultramarine blue, Prussian blue, cobalt blue, ferric blue, bismuth oxychloride, nacre, mica coated with titanium or with bismuth oxychloride, colored nacreous pigments such as titanium mica with iron oxides, titanium mica with, for example, ferric blue or chromium oxide, titanium mica with an organic pigment of the abovementioned type, and nacreous pigments based on bismuth oxychloride.

For example, iron oxide or titanium oxide pigments may be used.

The pigments may, for example, be treated with at least one hydrophobic agent to make them compatible with the organic phase of the emulsion. The at least one hydrophobic-treatment agent may be chosen from silicones, such as methicones, dimethicones and perfluoroalkylsilanes; fatty acids, such as stearic acid; metal soaps, for example, aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids and salts thereof; lecithin, and isopropyl triisostearyl titanate.

The N-acylamino acids may comprise at least one acyl group comprising from 8 to 22 carbon atoms, for example, chosen from a 2-ethylhexanoyl, caproyl, lauroyl, myristoyl, palmitoyl, stearoyl and cocoyl group. The salts of these compounds may be chosen from aluminium, magnesium, calcium, zirconium, zinc, sodium and potassium salts. The amino acid may, for example, be chosen from lysine, glutamic acid and alanine.

The term “alkyl” mentioned in the at least one hydrophobic agent above is an alkyl group comprising from 1 to 30 carbon atoms and, for example, comprising from 5 to 16 carbon atoms.

Hydrophobic-treated pigments are described, for example, in Patent Application No. EP-A-1 086 683, the disclosure relating to such pigments are incorporated herein by reference.

The liposoluble dyes may, for example, be chosen from Sudan Red, D&C Red No.17, D&C Green No. 6, β-carotene, soybean oil, Sudan Brown, D&C Yellow No.11, D&C Violet No. 2, D&C Orange No. 5, quinoline yellow, and annatto and bromo acids.

The water-soluble dyes may, for example, be chosen from beetroot juice, methylene blue and caramel.

The at least one dyestuff may be present in an amount ranging from 0.5% to 30% by weight, for example, ranging from 3% to 20% by weight and, further, for example, ranging from 5% to 15% by weight, relative to the total weight of the emulsion.

The water-in-oil solid emulsion disclosed herein may be a make-up composition, for example, provided in an form chosen from skin makeup compositions, such as a foundation, an eye shadow, a makeup rouge, a concealer product, a body makeup product and a lipstick. In one embodiment, the water-in-oil solid emulsion is a foundation composition.

The water-in-oil solid emulsion may be a care composition, for example, a skincare product chosen from skincare bases, care creams (day creams, night creams and anti-wrinkle creams) and makeup bases; lipcare compositions (lip balms); antisun-protection and self-tanning compositions; and deodorants.

The emulsion may comprise at least one other common cosmetic ingredient that may be chosen, for example, from hydrophilic and lipophilic gelling agents and thickeners, antioxidants, fragrances, preserving agents, neutralizers, sunscreens, vitamins, moisturizers, self-tanning compounds, anti-wrinkle active agents, emollients, hydrophilic and lipophilic active agents, free-radical scavengers, sequestering agents and film-forming agents.

Needless to say, a person skilled in the art will take care to select this or these optional compound(s), and/or the amount thereof, such that the advantageous properties of the emulsion disclosed herein is not, or are not substantially, adversely affected by the envisaged addition(s).

The emulsion disclosed herein may be prepared according to the following procedure:

The mixture of the constituents of the oily phase is first prepared by mixing together and heating, at a temperature ranging from 70° C. to 120° C., the waxes, the block copolymer of ethylene oxide and/or of propylene oxide and of an alkylene oxide comprising from 6 to 40 carbon atoms, and the non-volatile oils, followed by adding with stirring, at a temperature ranging from 60° C. to 80° C., the volatile oils, the fillers and the pigments.

The mixture of the constituents of the aqueous phase, comprising the water, the surfactants and the water-miscible solvents is prepared separately, with heating to a temperature ranging from 60° C. to 80° C.

Next, the aqueous phase is added to the oily phase, at a temperature ranging from 60° C. to 80° C. and the resulting mixture is stirred using a turbomixer until the water-in-oil emulsion is obtained. The emulsion is then poured into a container, for example a dish, and then cooled to room temperature until the solid emulsion is obtained.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary. depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements.

The various embodiments disclosed herein are illustrated in greater detail by the example described below, without, however, being limiting in nature.

### EXAMPLE 1

A solid foundation comprising the emulsion below was prepared:

Oily Phase:

| | |
|---|---|
| Copolymer of ethylene oxide (45 EO) and of epoxydodecane | 4 g |
| (22 mol) sold under the name “Elfacos ® ST 9” by the company | |
| Akzo Nobel | |
| Hydrogenated jojoba oil | 2.7 g |
| Ozokerite | 1 g |
| Isodecyl neopentanoate | 9.5 g |
| Cyclopentasiloxane | 22.5 g |
| Iron oxides coated with the disodium salt of aluminium stearoyl | 2.4 g |
| glutamate | |
| Titanium dioxide coated with the disodium salt of aluminium | 7.6 g |
| stearoyl glutamate | |
| Silica | 9.5 g |
| Polymethyl methacrylate | 3 g |
| Titanium dioxide nanopigments | 3 g |

Aqueous Phase:

| | |
|---|---|
| Water | 20 g |
| Butylene glycol | 3 g |
| Glycerol | 5 g |
| Magnesium sulphate | 1 g |
| Polyglyceryl-3 diisostearate | 0.3 g |
| Cetyl dimethicone copolyol (Abil ® EM 90 from the company | 2 g |
| Goldschmidt) | |
| Mixture of oxyethylenated oxypropylenated (18 EO/18 PO) | 2 g |
| polydimethylsiloxane, cyclopentasiloxane and water (10/88/2) | |
| (DC 2-5225 from Dow Corning) | |

The composition was prepared according to the following procedure:

The mixture comprising the waxes, the copolymer Elfacos ST 9 and the non-volitile oils was heated to 90° C. until a clear, uniform mixture was obtained; the volatile oils, the pigments and the fillers were then added with stirring, at 70° C. The mixture of the ingredients of the aqueous phase was then prepared with heating to 70° C., and the aqueous phase was then introduced at 70° C. into the oily phase, with stirring until the water-in-oil emulsion was obtained.

The emulsion was poured at 70° C. into a preheated dish and was then allowed to cool to room temperature until the solid emulsion was obtained.

A solid foundation with a hardness of 60 g, measured at 24 hours and at 20° C. according to the protocol described previously, was obtained, which has a specular surface (mirror effect) when the composition was disintegrated with the fingers; the foundation melted pleasantly on the skin when it was applied

What is claimed is:

1. A water-in-oil solid emulsion comprising an aqueous phase emulsified with at least one emulsifying surfactant, wherein said at least one emulsifying surfactant is in a fatty phase comprising:
at least one oil;
at least one wax; and
at least one block copolymer comprising at least one first monomeric unit chosen from ethylene oxide and propylene oxide units and at least one second monomeric unit chosen from alkylene oxide units comprising from 6 to 40 carbon atoms, wherein the at least one block copolymer has a weight-average molecular weight ranging from 5000 g/mol to 8000 g/mol and
wherein the emulsion has a hardness such that the penetration force is greater than or equal to 40 g.

2. The emulsion according to claim 1, wherein the alkylene oxide units of the at least one block copolymer comprise from 6 to 30 carbon atoms.

3. The emulsion according to claim 2, wherein the alkylene oxide units of the at least one block copolymer comprise from 8 to 20 carbon atoms.

4. The emulsion according to claim 3, wherein the alkylene oxide units of the at least one block copolymer comprise from 10 to 18 carbon atoms.

5. The emulsion according to claim 4, wherein the alkylene oxide units of the at least one block copolymer comprise from 10 to 14 carbon atoms.

6. The emulsion according to claim 1, wherein the at least one block copolymer has a weight-average molecular weight ranging from 5500 g/mol to 7000 g/mol.

7. The emulsion according to claim 6, wherein the at least one block copolymer has a weight-average molecular weight ranging from 5500 g/mol to 6500 g/mol.

8. The emulsion according to claim 7, wherein the at least one block copolymer has a weight-average molecular weight ranging from 5800 g/mol to 6200 g/mol.

9. The emulsion according to claim 1, wherein the at least one block copolymer comprises from 35 to 55 first monomeric units chosen from ethylene oxide and propylene oxide units and from 15 to 30 second monomeric units chosen from alkylene oxide units comprising from 6 to 40 carbon atoms.

10. The emulsion according to claim 1, wherein the at least one block copolymer is such that the ratio of the number of first monomeric units to the number of second monomeric units ranges from 1.5:1 to 2.5:1.

11. The emulsion according to claim 10, wherein the at least one block copolymer is such that the ratio of the number of first monomeric units to the number of second monomeric units ranges from 1.8:1 to 2.3:1.

12. The emulsion according to claim 11, wherein the at least one block copolymer is such that the ratio of the number of first monomeric units to the number of second monomeric units ranges from 1.9:1 to 2.1:1.

13. The emulsion according to claim 1, wherein the at least one block copolymer is present in an amount ranging from 1% to 25% by weight, relative to the total weight of the emulsion.

14. The emulsion according to claim 13, wherein the at least one block copolymer is present in an amount ranging from 1% to 18% by weight, relative to the total weight of the emulsion.

15. The emulsion according to claim 14, wherein the at least one block copolymer is present in an amount ranging from 1% to 10% by weight, relative to the total weight of the emulsion.

16. The emulsion according to claim 1, wherein the emulsion has a hardness such that the penetration force ranges from 40 g to 150 g.

17. The emulsion according to claim 16, wherein the emulsion has a hardness such that the penetration force ranges from 50 g to 120 g.

18. The emulsion according to claim 17, wherein the emulsion has a hardness such that the penetration force ranges from 50 g to 90 g.

19. The emulsion according to claim 18, wherein the emulsion has a hardness such that the penetration force ranges from 60 g to 80 g.

20. The emulsion according to claim 1, wherein the at least one oil in the fatty phase is chosen from volatile oils.

21. The emulsion according to claim 20, wherein the volatile oils are chosen from at least one of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, isododecane, isodecane and isohexadecane.

22. The emulsion according to claim 21, wherein the volatile oils are present in an amount ranging from 0.1% to 35% by weight, relative to the total weight of the emulsion.

23. The emulsion according to claim 22, wherein the volatile oils are present in an amount ranging from 1% to 30% by weight, relative to the total weight of the emulsion.

24. The emulsion according to claim 23, wherein the volatile oils are present in an amount ranging from 5% to 25% by weight, relative to the total weight of the emulsion.

25. The emulsion according to claim 1, wherein the fatty phase comprises at least one oil chosen from non-volatile oils.

26. The emulsion according to claim 25, wherein the non-volatile oils are present. in an amount ranging from 0.1% to 35% by weight, relative to the total weight of the emulsion.

27. The emulsion according to claim 28, wherein the non-volatile oils are present in an amount ranging from 1% to 30% by weight, relative to the total weight of the emulsion.

28. The emulsion according to claim 27, wherein the non-volatile oils are present in an amount ranging. from 5% to 25% by weight, relative to the total weight of the emulsion.

29. The emulsion according to claim 1, wherein the at least one wax has a hardness ranging from 5 MPa to 9 MPa.

30. The emulsion according to claim 29, wherein the at least one wax has a hardness ranging from 6 MPa to 9 MPa.

31. The emulsion according to claim 30, wherein the at least one wax has a hardness ranging from 7 MPa to 9 MPa.

32. The emulsion according to claim 1, wherein the at least one wax is chosen from carnauba wax, microcrystalline waxes, ozokerites, hydrogenated jojoba oil, polyethylene waxes, poly(C₂₄-C₂₈)alkylmethyldimethylsiloxane waxes, palm butter, C₂₀-C₄₀ alkyl stearate, stearyl benzoate, and shellac wax.

33. The emulsion according to claim 32, wherein the. at least one wax is chosen from carnauba wax, ozokerites, hydrogenated jojoba oil and polyethylene waxes.

34. The emulsion according to claim 1, wherein the at least one wax is present in an amount ranging from 1% to 10% by weight, relative to the total weight of the emulsion.

35. The emulsion according to claim 34, wherein the at least one wax is present in an amount ranging from 2% to 7% by weight, relative to the total weight of the emulsion.

36. The emulsion according to claim 35, wherein the at least one wax is present in an amount ranging from 2.5% to 5% by weight, relative to the total weight of the emulsion.

37. The emulsion according to claim 1, wherein the aqueous phase is present in an amount ranging from 30% to 50% by weight, relative to the total weight of the emulsion.

38. The emulsion according to claim 37, wherein the aqueous phase is present in an amount ranging from 35% to 45%, by weight relative to the total weight of the emulsion.

39. The emulsion according to claim 1, wherein the aqueous phase comprises at least one water-miscible solvent chosen from C₂-C₄ primary alcohols, glycols and glycol ethers.

40. The emulsion according to claim 1, wherein the at least one emulsifying surfactant is chosen from fatty acid esters of polyols; alkyl and alkoxy dimethicone copolyols comprising at least one chain chosen from alkyl and alkoxy chains, wherein the at least one chain is pendent or at the end of a silicone skeleton; and polyoxyalkylenated fafty acid esters of glycol.

41. The emulsion according to claim 40, wherein the fatty acid esters of polyols are chosen from glyceryl and sorbitol mono-, di-, tri- and sesquioleates and stearates, and glyceryl and polyethylene glycol laurates.

42. The emulsion according to claim 40, wherein the at least one chain comprises from 6 to 22 carbon atoms.

43. The emulsion according to claim 40, wherein the at least one emulsifying surfactant is chosen from C₈-C₂₂ alkyl dimethicone copolyols of formula (I) below: 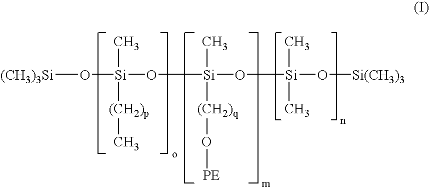
wherein:
PE is chosen from groups (—C₂H₄O)ₓ—(C₃H₆O)_{y}—R, wherein R is chosen from a hydrogen atom and alkyl radicals comprising from 1 to 4 carbon atoms, x is a number ranging from 0 to 100 and y is a number ranging from 0 to 80, provided that x and y are not simultaneously equal to 0;
m is a number ranging from 1 to 40;
n is a number ranging from 10 to 200;
o is a number ranging from 1 to 100;
p is a number ranging from 7 to 21; and
q is a number ranging from 0 to 4.

44. The emulsion. according to claim 1, wherein the at least one emulsifying surfactant is present in an amount ranging from 1% to 10% by weight, relative to the total weight of the emulsion.

45. The emulsion according to claim 44, wherein the at least one emulsifying. surfactant is present in an amount ranging from 2% to 5% by weight, relative to the total weight of the composition.

46. The emulsion according to claim 1, further comprising at least one filler.

47. The emulsion according to claim 1, further comprising at least one dyestuff.

48. The emulsion according to claim 1, further comprising at least one cosmetic ingredient chosen from hydrophilic and lipophilic gelling agents and thickeners, antioxidants, fragrances, preserving agents, neutralizers, sunscreens, vitamins, moisturizers, self-tanning compounds, anti-wrinkle active agents, emollients, hydrophilic and lipophilic active agents, free-radical scavengers, sequestering agents and film-forming agents.

49. The emulsion according to claim 1, wherein the emulsion is provided in a form chosen from foundations, eye shadows, makeup rouges, concealer products, body makeup products, lipsticks, skincare bases, care creams; lipcare compositions; antisun compositions, self-tanning compositions; and deodorants.

50. A foundation comprising a water-in-oil solid emulsion comprising an aqueous phase emulsified with at least one emulsifying surfactant, wherein said at least one emulsifying surfactant is in a fatty phase comprising:
at least one oil;
at least one wax; and
at least one block copolymer comprising at least one first monomeric unit chosen from ethylene oxide and propylene oxide units and at least one second monomeric unit chosen from alkylene oxide units comprising from 6to 40 carbon atoms, wherein the at least one block copolymer has a weight-average molecular weight ranging from 5000 g/mol to 8000 g/mol and
wherein the emulsion has a hardness such that the penetration force is greater than or equal to 40 g.

51. A non-therapeutic cosmetic makeup and/or care process for the skin comprising applying to the skin at least one composition comprising a water-in-oil solid emulsion comprising an aqueous phase emulsified with at least one emulsifying surfactant, wherein said at least one emulsifying surfactant is in a fatty phase comprising:
at least one oil;
at least one wax; and
at least one block copolymer comprising at least one first monomeric unit chosen from ethylene oxide and propylene oxide units and at least one second monomeric unit chosen from alkylene oxide units comprising from 6 to 40 carbon atoms, wherein the at least one block copolymer has a weight-average molecular weight ranging from 5000 g/mol to 8000 g/mol and
wherein the emulsion has a hardness such that the penetration force is greater than or equal to 40 g.
